(19) United States
(12) Patent Application Publication
(10) Pub. No.: US 2022/0189618 A1
(43) Pub. Date: Jun. 16, 2022

(54) IN-WORKFLOW ARTIFICIAL INTELLIGENCE (AI)-ENABLED INTERRUPTION HANDLING FOR DIAGNOSTIC RADIOLOGY

(71) Applicant: KONINKLIJKE PHILIPS N.V., EINDHOVEN (NL)

(72) Inventors: PRESCOTT PETER KLASSEN, CAMBRIDGE, MA (US); MICHAEL PROKLE, BOSTON, MA (US)

(21) Appl. No.: 17/441,300

(22) PCT Filed: Mar. 20, 2020
(86) PCT No.: PCT/EP2020/057756

§ 371 Date: Sep. 21, 2021

Related U.S. Application Data
(60) Provisional application No. 62/822,121, filed on Mar. 22, 2019.

(51) Int. Cl. G16H 40/20; G06N 5/02; G16H 30/20; H04M 3/436; H04M 3/523

(52) U.S. Cl. CPC G16H 40/20; G06N 5/022; H04M 3/5232; H04M 3/436; G16H 30/20

(57) ABSTRACT

A non-transitory computer readable medium (26) stores instructions executable by at least one electronic processor (20) to perform a request resolution method (100). The method includes: intercepting (102) communication requests (31) directed to a radiology department; classifying (104) the communication requests; assigning (106) the communication requests to agent queues of a plurality of agent queues (40) based on at least the classifications of the communication requests; and routing (108) the communication requests assigned to each agent queue to a request resolution agent (50) corresponding to the agent queue.

### FIELD

The following relates generally to the radiology arts, radiology workstation arts, radiology interpretation arts, radiology request allocation arts, and related arts.

### BACKGROUND

Radiology departments provide services for many (often most or all) clinical disciplines at a hospital or other medical institution. As such, a radiologist typically handles a heavy workload, and routinely deals with a wide range of different types of clinicians with varying knowledge of radiology. A radiologist often also has supervisory duties, for example providing support expertise for imaging technicians who operate magnetic resonance imaging (MRI) scanners, computed tomography (CT) scanners, positron emission tomography (PET) scanners, and/or imaging devices of other imaging modalities.

One of the main measures of productivity in radiology is the volume of reads, where a “read” corresponding to reviewing, and preparing a radiology report on, a radiology examination of a single patient. While performing readings of radiology examinations may be the primary duty of a radiologist during a work shift, the radiologist is also expected to handle calls from physicians, imaging technicians, and others. These calls can be a substantial burden on the radiologist's time, and can break the radiologist's concentration when performing a complex reading. In some recent studies, radiologists were found to be interrupted every 4-12 minutes during regular business hours. See, e.g., A. Schemmel et al., “Radiology workflow disruptors: a detailed analysis,” Journal of the American College of Radiology, vol. 13, no. 10, pp. 1210-1214, 2016); R. M. Ratwani et al., “A human factors approach to understanding the types and sources of interruptions in radiology reading rooms,” Journal of the American College of Radiology, vol. 13, no. 9, pp. 1102-1105, 2016; T. Drew et al., “Quantifying the costs of interruption during diagnostic radiology interpretation using mobile eye-tracking glasses,” Journal of Medical Imaging, vol. 5, no. 3, p. 031406, 2018.). After hours, interruptions occur even more frequently, with on-call radiologists receive an average of 72 phone calls during a typical 12 hour shift (see, e.g., J. Y. John-Paul, A. P. Kansagra, and J. Mongan, “The radiologist's workflow environment: evaluation of disruptors and potential implications,” Journal of the American College of Radiology, vol. 11, no. 6, pp. 589-593, 2014.).

Interruptions, either in-person or by-phone, affect a radiologist's ability to efficiently read radiology examinations so as to maintain both high accuracy and high throughput. This not only directly influences the radiologist's measure of productivity but may also have impact on patients' wait time for a diagnosis and the expected health outcome. Similarly, these interruptions may also affect radiologists' attention and quality of the read, potentially affecting patients' health treatment.

The following discloses new and improved systems and methods to overcome these problems.

### SUMMARY

In one disclosed aspect, a radiology request monitoring system includes at least one electronic processor; and a non-transitory computer readable medium storing instructions executable by the at least one electronic processor. The instructions include: instructions implementing a radiology reading environment via which radiology images are displayed on the display device and via which a radiology report is received via the one or more user input devices; instructions implementing a communication requests interface configured to intercept communication requests directed to a user of the radiology reading environment; instructions implementing an interpreter module configured to classify the intercepted communication requests; instructions implementing a scheduler module configured to assign the communication requests to agent queues of a plurality of agent queues based on at least the classifications of the communication requests; and instructions implementing a dispatcher module configured to route the communication requests assigned to each agent queue to a request resolution agent corresponding to the agent queue.

In another disclosed aspect, a non-transitory computer readable medium stores instructions executable by at least one electronic processor to perform a request resolution method. The method includes: intercepting communication requests directed to a radiology department; classifying the communication requests; assigning the communication requests to agent queues of a plurality of agent queues based on at least the classifications of the communication requests; and routing the communication requests assigned to each agent queue to a request resolution agent corresponding to the agent queue.

In another disclosed aspect, a request resolution method includes: implementing a radiology reading environment via which radiology images are displayed on a display device of a workstation and via which a radiology report is received via one or more user input devices of the workstation; intercepting, with a communication requests interface, communication requests directed to a user of the radiology reading environment; classifying, with an interpreter module, the intercepted communication requests; assigning, with a scheduler module, the communication requests to agent queues of a plurality of agent queues based on at least the classifications of the communication requests, routing, with a dispatcher module, the communication requests assigned to each agent queue to an AI-enabled dialog expert system corresponding to the agent queue; and monitoring, with an AI optimization module, processing of the communication requests by the AI-enabled dialog expert system.

One advantage resides in limiting interruptions of a radiologist during radiology reading sessions.

Another advantage resides in screening interruptions of a radiologist during radiology reading sessions according to factors such as modality, request type, and person requesting categories.

Another advantage resides in prioritizing request types by a set of priority rules for a radiologist during radiology reading sessions.

Another advantage resides in automating requests of a radiologist during radiology reading sessions.

Another advantage resides in directing requests to appropriate manual or automated resolution channels based on request content, source, and/or other factors.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows a workflow schedule monitoring system according to one aspect.

FIG. 2 shows exemplary flow chart operations of the system of FIG. 1.

### DETAILED DESCRIPTION

Radiologists typically perform readings of radiology examinations in a dedicated “reading room” containing radiology workstations each having suitable high-resolution graphical display monitors for presenting radiology images, and running reading software for enabling the radiologist to review images (with zoom, pan, et cetera) and to write up (or dictate) radiology reports summarizing findings. Radiologist productivity is closely monitored, with radiologists expected to meet time and/or throughput goals for each work shift. At the same time, the radiologist must maintain a high level of accuracy and comprehensiveness in capturing salient medical findings from imaging examinations.

Interruptions in the form of telephone calls, emails, text messages, and the like are a significant problem for radiologists. These can be from imaging technologists asking for clarification regarding a current imaging examination being performed; doctors calling to ask for clarification or other information about radiology reports they have received; other radiologists asking for advice; and so forth. By some estimates, a radiologist may receive around 6 interruptions every hour. Although frequent, these interruptions are typically not life-critical, as the radiologist is not an emergency care provider.

The following discloses an interrupt handling system that intercepts and characterizes interruptions, prioritizes them, and to the extent practicable automatically resolves interruptions by way of a knowledge base (KB) or an artificial intelligence (AI) chat bot so as to reduce the number of interruptions that must be handled by a radiologist. The interruption handling system includes an interpreter that classifies an interruption, a scheduler that contextualizes the interruption, and a dispatcher that dispatches the interruption to the appropriate resolution entity, such as the aforementioned KB and/or AI chat bot, a call or message queue for a radiology specialist group (e.g. MM radiology experts), a call or message queue for a specific radiologist, and/or so forth.

The interpreter assigns standardized attributes to the interruption, such as caller attributes describing the caller; request attributes describing the type of request (e.g. pertaining to a current examination, or to a previously prepared radiology report, etc.); a timestamp of when a call was received; the patient who is the subject of the call; the intended recipient (e.g. a named radiologist or a general question characterized by anatomy, imaging modality, reason for exam, etc.); interruption complexity (e.g. number of questions being asked), and/or so forth. This information may be variously gathered, e.g. by applying natural language processing (NLP) to the text of an email or text message (or, in the case of a voice call, to text generated by automatic transcription of the voice message), and/or information gathered from the caller using an automated call receiving agent (for example, executing an automated dialog script to obtain specific information such as caller name, patient name, radiology examination identifier if available, and/or so forth). The collected information is formulated into a canonical representation of the request, for example using a vector or list of attributes whose values are determined (to the extent possible) from the gathered information.

The scheduler receives the canonical request represented by the values of the attributes assigned by the interpreter, and augments this with context information such as the current queue lengths for the various radiologists, radiologist pools, and for the AI chat bot and/or the KB (if one or both of these are available), along with statistical information such as an expected call time and expected wait time for each resolution path. The dispatcher then queues the interruption into an appropriate resolution channel (e.g. KB, AI chat bot, radiologist pool, or a named radiologist channel) based on the canonical request and augmented context.

Another aspect is to enable radiologist configuration of the system. For example, a radiologist who wants to work on CT imaging examinations for the next two hours can assign him/herself to the CT imaging pool and so may be called upon by the system to handle interruptions relating to CT (but not to other modalities such as MRI). This helps to focus a radiologist on specific modality and/or anatomy for extended time intervals so as to improve efficiency/accuracy. The radiologist may also set an interruption level, e.g. do not interrupt at all, or do not interrupt for more minor matters.

In one contemplated implementation, the call management system can be a separate always-on system running on the radiology reading workstation (and/or optionally on a network computer or computer cluster operatively connected with the workstation) in parallel with the radiology reporting software. The call management system is configured to intercept phone calls (e.g. using Voice-Over Internet Protocol (VOIP) calling), emails (e.g. via a plug-in running on the hospital's email program, e.g. Outlook), and internal text messages (e.g. via an SMS, SMTP, or other text messaging plug-in running on the radiology reporting system or on an ancillary text messaging system). Notifications or alerts can be provided via a notifications window of the call management program, and/or via a headless structure in which the notifications are presented via a plug-in integrated with the radiology reporting system.

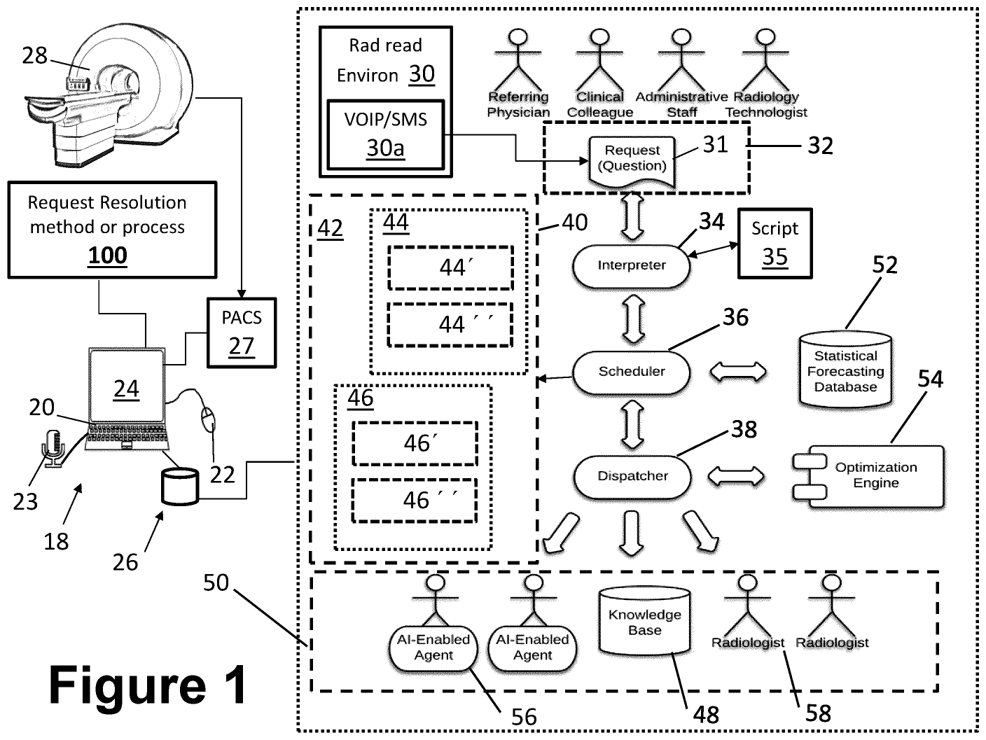

With reference to FIG. 1, an illustrative radiology workstation environment including a radiology request monitoring system is shown. As shown in FIG. 1, the radiology request monitoring system includes a computing device 18 (e.g., typically a radiology workstation computer, or more generally a computer, although another form factor such as a tablet, a smartphone, and so forth is also contemplated). The workstation 18 comprises a computer or other electronic data processing device with typical components, such as at least one electronic processor 20, at least one user input device (e.g., a mouse, a keyboard, a trackball, and/or the like) 22, the user input device(s) also commonly including a dictation microphone 23 used by a radiologist in dictating a radiology report, and a display device 24. In some embodiments, the display device 24 can be a separate component from the computer 18. The radiology workstation 18 typically includes at least one high resolution display device 24 for displaying radiology images in high fidelity, and some radiology workstations may include two displays (second display not shown) with one used to display high resolution images and the other used to display the radiology report and/or other text-based information.

The workstation 18 can also include one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth. The workstation 18 also accesses a Picture Archiving and Communication System (PACS) 27 and/or other radiology-related database(s) such as a Radiology Information System (RIS), combined PACS/RIS system and/or so forth. For illustrative purposes, the PACS 27 is shown, but it is to be understood that this more generally represents a radiology-related information system/database storing at least radiology examinations. Typically, the data for each radiology examination stored in the PACS 27 includes the radiology images acquired of the patient undergoing the examination using an illustrative magnetic resonance imaging (MRI) scanner 28 along with relevant metadata such as patient identifier (PID), name of ordering physician, a reason for examination, imaging parameters used in acquiring the images, and/or so forth. The display device 24 is configured to display the acquired radiology images and relevant metadata, along with the radiology report-under-draft, retrieved prior radiology reports on the patient (which may be reviewed by the radiologist during drafting of the radiology report for the current radiology examination), and/or so forth.

The radiology workstation 18 is configured to receive requests from one or more sources (e.g., a physician, a nurse, a technician, a radiologist, an administrative staff member, and the like). These requests can be in any suitable form, including email, phone call, text messages, internal messaging systems, and so forth. The radiology workstation 18 stores instructions to process and route the requests to an appropriate mechanism for completion.

In another embodiment, the computing device 18 includes a separate command station (e.g., a server computer). The server 18 is configured for handling incoming requests from radiologists who are logged in to workstations dispersed about a hospital. The server 18 is also connected to the different workstations to get local information of that workstation based on the radiologist that is logged in.

As shown in FIG. 1, the non-transitory storage media 26 stores instructions 30, 32, 34, 36, 38 executable by the at least one electronic processor 20. The instructions include instructions which implement a radiology reading environment 30 via which radiology images are displayed on the display device 24 and via which a radiology report is received via the one or more user input devices 22, 23. By way of non-limiting illustrative example, one contemplated radiology reading environment is the radiology reading environment provided by the Philips IntelliSpace™ PACS system. The radiology reading environment 30 provides for retrieval of images of a radiology examination from the PACS 27, display of the images, user-directed manipulation of the images (e.g., zoom, pan, apply image filters, mark/dimension image features such as lesions using on-screen cursors controlled by the mouse or other user input device 22, or so forth), and receipt of the radiology report via the dictation microphone 23 and/or other user input device 22; as well as providing for retrieval of prior radiology reports on the patient or other patient information that may be of use to the radiologist in performing a comprehensive reading of a radiology examination. The radiology reading environment 30 still further provide, or has access to, one or more communication interfaces 30a, such as a Voice Over Internet Protocol (VOIP) telephonic interface; an SMS, SMTP, or other text messaging interface; an email client; and/or so forth. The illustrative communication interface(s) 30a is shown in diagrammatic FIG. 1 as a component of the radiology reading environment 30; however, it is additionally or alternatively contemplated for one or more communication interface(s) to be a separate module (e.g. a VOIP client or SMS client that is separate from the radiology reading environment) that is linked with the radiology reading environment by way of a hook, plug-in, add-on module, or the like. The communication interface(s) 30a are operatively connected with the Internet and/or another electronic network to receive communication requests in the form of telephone calls, text messages, emails, or so forth. The radiology reading environment 30 provides a communication configuration user interface via which the user of the radiology reading environment configures communication preferences relating to a maximum number of communication requests allowed per unit time, and prioritization of communication requests on the basis of request content including, for example, one or more of type of modality and type of anatomy.

Conventionally, messages received at the communication interface(s) 30a would be brought to the attention of the radiologist in a more or less intrusive manner. For example, a VOIP telephone call might trigger an audible ringtone notifying the radiologist of the incoming telephone call, while an email or text message alert may be in the form of an audible beep sound. In some conventional communication interfaces, the user can elect to silence the ringtone, beep, or other notification, with the call going to voicemail in the case of a telephonic message. But, while this results in the radiologist being undisturbed by incoming communication requests, it also means those requests go unanswered until the radiologist elects to check his or her voice messages, text messages, electronic mailbox, or the like. In the illustrative embodiment of FIG. 1, by contrast, the instructions also implement a communication requests interface 32 configured to intercept communication requests 31 received via the communication interface(s) 30a and directed to a user of the radiology reading environment 30. As used herein, the term “intercept” (and variants thereof) includes preventing the communication requests 31 from being immediately presented to the user without processing or queuing by the remaining components of the message handling system described below.

The instructions also implement an interpreter module 34 configured to classify the communication requests 31 intercepted by the communication requests interface 32. To do so, the interpreter module accepts communication requests 31 from the communication requests interface 32 and classifies them by analyzing each request for attributes that are used to sort, classify, and route as values for attributes of an attribute vector. The vector is a standardized representation using a set of standard attributes to allow for easy comparison between classifications of different communication requests 31. These attributes may, by way of non-limiting example, include one or more of the following: one or more caller attributes describing the caller including the identity of caller; one or more request type attributes describing the type of request including a request attribute pertaining to a current examination, and a previously prepared radiology report; a timestamp attribute storing a time when the communication request was intercepted; a patient attribute identifying a patient to which the communication request pertains; at least one request content attribute indicating one or more of anatomy, imaging modality, or reason for exam content of the communication request; and an interruption complexity attribute whose value is determined based at least on a number of questions being asked in the communication request 31; an urgency attribute storing an indication of urgency of the message; and/or so forth.

In some embodiments, the interpreter module 34 may employ an interactive script 35 to acquire some of the information for ascertaining values for certain attributes of the communication request 31. For example, in the case of a VOIP telephone call, the script 35 may employ an electronic call agent to play back questions (for example, “What is the patient identifier?” and receive telephonic responses which are processed by transcription software to extract the information (e.g. the PID). As another example, an automatic SMS agent can similarly query the requestor via follow-up text message(s) to obtain such information. The value of the urgency attribute for a message may be set based on an affirmative urgency value provided by the requestor, e.g. in response to a question of urgency presented in the script 35, or may be determined based on information extracted from the message or based on other attributes (for example, a communication request whose requestor is an imaging technician and whose content includes a request for information about a specific setting for a radiology examination currently in progress may be given a higher urgency value than an information request sent by a physician regarding a previously-performed radiology reading). Other factors for setting the urgency value may include reason for examination (for example, a routine screening examination will be lower priority than an examination intended to diagnose a critically ill patient) or so forth.

A sorting and classification algorithm executed by the interpreter module 34 analyzes the values of the attributes including full text analysis of the body of the request, in order to create a canonical representation of the communication request including values for attributes such as priority, imaging modality type, clinical question, and other data that is relevant to scheduling and dispatching of the communication request. For example, the interpreter module 34 can classify the communication requests based on imaging modality, or type of communication (e.g., a text-message directed to a specific radiologist, a phone call from a technician, or an email from a referring physician, etc.).

In some examples, the interpreter module 34 is configured to assign the classifications to the communication requests at least based on natural language processing of natural language content of the communication requests. This text can be text directly extracted from a text message, or text generated by voice recognition (i.e. transcription) software in the case of a telephone call.

The instructions also implement a scheduler module 36 configured to assign the communication requests to agent queues of a plurality of agent queues 40 for request resolution based on at least the classifications of the communication requests by the interpreter module 34. The request resolution agents may, for example, include a knowledge base (KB) agent, an artificial intelligence (AI) chatbot, a voicemail queue for a specific radiologist, a voicemail queue for a radiology group (e.g., all radiologists with MRI expertise), a text message queue for a specific radiologist or for a group of radiologists, an electronic mailbox for a specific radiologist or a group of radiologists, an immediate connection to the radiologist via a telephonic interface (e.g. activating a ringtone to alert the radiologist and providing for telephonic communication via the dictation microphone 23 and a loudspeaker (not shown) of the workstation 18), an immediate connection to the radiologist by immediately opening a text messaging window on the display 24; an immediate pop-up message to all members of a group of radiologists asking that an urgent communication request be handled (followed by telephonic or messaging window connection upon acceptance of the pop-up message by a member of the group); and/or so forth.

The scheduler module 36 schedules the requests to specific queues based on criteria such as (by way of non-limiting example): imaging modality, type of communication, urgency, and expected time-to-complete. The scheduler module 36 can send messages to different agent queues 40 to create a dispatch request. In some embodiments, the plurality of agent queues 40 includes at least one user interface queue 42 whose corresponding request resolution agent comprises a user interface 44 with the user of the radiology reading environment 30. The user interface 44 can be, for example, a telephonic (e.g., VOIP) interface 44′ or a text messaging user interface 44″ with the user of the radiology reading department 30. The plurality of agent queues 40 further includes at least one automated agent queue 46 whose corresponding request resolution agent comprises an automated request resolution agent. In some embodiments, the at least one automated agent queue 46 includes, for example, an automated query queue 46′ whose corresponding request resolution agent comprises an expert system retrieving responses from a technical knowledge database 48, and/or an AI-enabled dialogue window queue 46″ whose corresponding request resolution agent comprises an AI-enabled dialog expert system interfacing with the technical knowledge database 48. The technical knowledge database 48 includes data related to the technology of the request (e.g., echo time for MRI brain scans).

The scheduler module 36 is also configured to analyze the criteria, including modality, type of communication, urgency, and expected time-to-complete, and cluster the data to determine efficiency parameters. For example, the scheduler module 36 analyzes trends in the criteria data, such as a balance of a quick response requirement with a longer response requirement of the requests, how often requests are made on a specific type of imaging modality (e.g., a number of MR-related requests, CT-related requests, and so forth), how often requests are made for a particular topic (e.g., a patient couch on an imaging device, how often detector modules are malfunctioning on an imaging device, and so forth); how often requests are made for information on a particular patient, and so forth. The scheduler module 36 analyzes these trends to cluster this data, which can be used to direct the request to a specific resolution agent 50 via the dispatcher module 38.

The instructions also implement a dispatcher module 38 configured to route the communication requests assigned to each agent queue 40 by the scheduler module 36 to a request resolution agent 50 corresponding to the agent queue. The dispatcher module 38 removes dispatch requests from the queues 40 and determines how to route to the appropriate request resolution agent 50 (e.g., a simple query to knowledge base, a request for an AI-enabled agent trained to manage the request, or a human radiologist specifically or simply next in queue for a request, and so forth). In the case of a request whose response time is not time-critical, such as a communication request that is queued to go to voicemail or an electronic mailbox, the dispatch may entail sending the message to the radiologist's voicemail or electronic mailbox. In the case of an automated agent such as a KB 46′ or AI chatbot 46″, such an agent can generally handle multiple communication requests concurrently, and so the dispatch entails initiating a KB data retrieval or AI chatbot session. For interfaces with human agents to provide an immediate response (e.g. the VOIP telephonic connection 44′ or a messaging window pop-up 44″), if that agent is already occupied (e.g., the radiologist is currently handling another VOIP call) then the dispatch may place the requestor on hold and provide a notification on the workstation 18 to notify the radiologist of the incoming VOIP call, text message, or other urgent communication request which is on hold.

In some embodiments (or for queues which are not time-critical, such as handling a VOIP phone call by sending it to the radiologist's voicemail), the scheduler module 36 receives the requests from the interpreter module 34 and immediately dispatches the requests. In this embodiment (or for these non-time-critical queues), no actual scheduling occurs. The requests are merely routed to the user interface queues 42 of the agent queues 40 where they are processed on a first-in, first-out basis. Alternatively, for more intrusive agents such as the VOIP interface 44′ or a text messaging user interface 44″, the scheduler module 36 actually assigns scheduled times, especially for the user interface queues 42 where the radiologist may, for example, want to set a “do not disturb” status for a desired time period for non-critical requests. For example, the agent queues 40 can include a user interface queue 42 for critical requests that cannot be delayed, and a user interface 42 for non-critical requests that can be delayed. In another example, the agent queues 40 can include a user interface queue 42 for MRI-related requests, and a user interface 42 for CT-related requests. For these cases, the scheduler module 36 receives the requests from the interpreter module 34 and combines the classifications of the requests with context stored in a statistical forecasting knowledge database 52 to schedule the routing of the communication tasks by the dispatcher module 38.

To facilitate the scheduling of routing of more intrusive communication requests, such as to the VOIP interface 44′ or a text messaging user interface 44″, the instructions optionally further implement an AI optimization module 54 configured to: (i) monitor processing of the communication requests by the request resolution agents; and (ii) update the forecasting data of the statistical forecasting knowledge database 52 on the basis of the monitored processing of the communication requests by the request resolution agents. The AI optimization module 54 tracks real-time workload of all agents and provides the logic & statistical forecasting (e.g., expected wait time for a radiologist to finish) to the dispatcher module 38 to prioritize requests. For example, an Operations Research and queuing theory can be used to allocate requests to virtual waiting line as well as reprioritizing requests.

The context stored in the statistical forecasting knowledge database 52 may, for example, include: forecasting data including one or more of: current queue lengths for the queues of the plurality of queues, expected wait times for the queues of the plurality of queues, and historical wait times for the queues of the plurality of queues, historic processing times, current queues, pool of radiologists and other available resources. The combined record is then sent to the dispatcher module 38. The dispatcher module 38 analyzes the combined request and context from the technical knowledge database 48, and determines, based on properties of the request (e.g., clinical question, priority, etc.) if the request can be handled by a simple knowledge base query 52, dialogue with an AI-enabled dialog expert system 56 trained for handling specific types of requests, or an actual human radiologist 58.

If the request is determined to be directed to a particular radiologist or to a particular group of radiologists (e.g. the MRI specialists), then the optimization module re-prioritizes the current queue based on availability of the radiologist or radiologists, and their preferences. Radiologists can interact with the dispatcher module 38, by uploading their preferences for communication and their availability. During the entire end-to-end process, the original requestor is continuously updated with estimated wait times and status of their request.

The AI optimization module 54 also tracks a changing availability of the radiologists based on data submitted by the radiologists via the dispatcher module 38. To do so, the AI optimization module 54 tracks how often a particular radiologist needs to be available based on the preferences submitted by the group of radiologists via the dispatcher module 38. For example, a particular radiologist may upload a request via the dispatcher module 38 to not be disturbed for two hours. The AI optimization module 54 analyzes this request in conjunction with the other requests made by the other radiologists, and determines where the request by the particular radiologist needs to be changed based on the availability of the other radiologists (e.g., the particular radiologist may not be able to have the “two hour do-not-disturb” request granted because too many other radiologist have made a similar two hour-long request). In another example, the AI optimization module 54 determines a maximum time period for which a “do not disturb” request that can be selected. That is, on days with a large number of a requests, a smaller time window (e.g., 2 hours) can be a maximum allowed “do not disturb” request, while on days with a smaller number of requests, a larger time window (e.g., 4 hours) can be a maximum allowed “do not disturb” request.

The scheduler module 36 can also serve as an information packaging module for presenting information to a radiologist. For example, for communication requests that are assigned to human radiologist(s) 58, the scheduler module 36 can retrieve information related to the request (e.g., modality type, a request for a particular radiologist, a time frame for the request, and so forth) and determine what type of information would be required by the radiologist to answer the request (e.g., based on information retrieved from the database 52). A request package including the request, along with the retrieved information, can be displayed on the display device 24 as, for example, natural language text, pictures, hyperlinks, and so forth.

The communication requests management system is configured as described above to perform a request resolution method or process 100. The non-transitory storage medium 26 stores instructions which are readable and executable by the at least one electronic processor 20 of the workstation 18 and to perform disclosed operations including performing the request resolution method or process 100. In some examples, the method 100 may be performed at least in part by cloud processing.

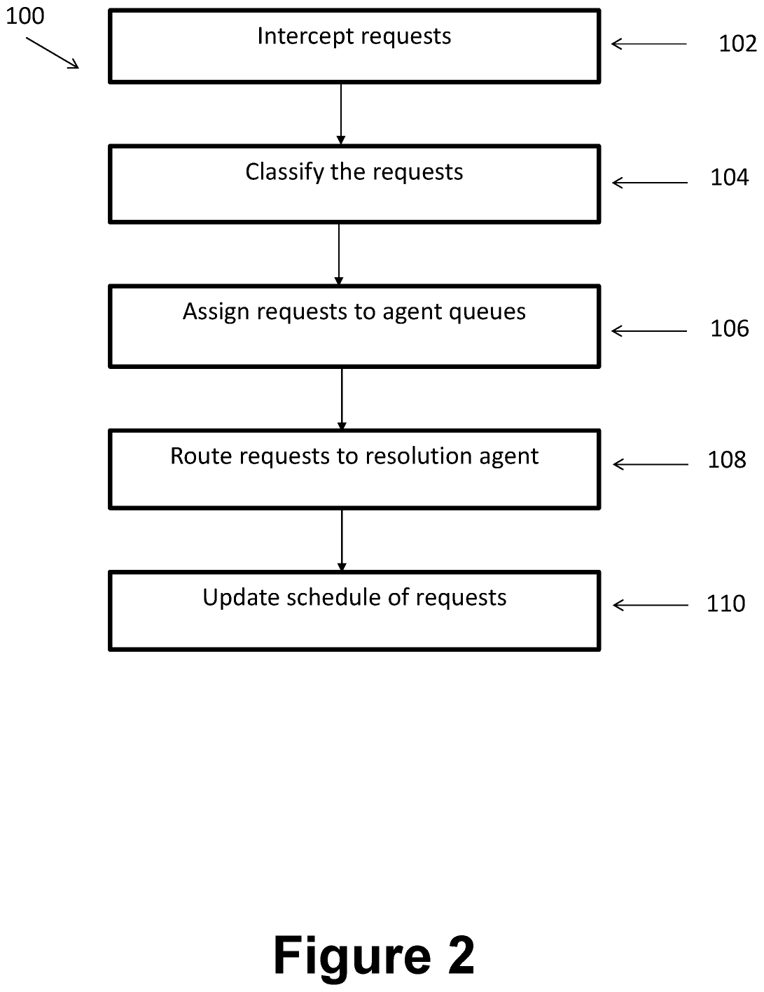

With reference to FIG. 2, an illustrative embodiment of the request resolution method 100 is diagrammatically shown as a flowchart. At 102, the communication requests interface 32 is configured to intercept communication requests 31 directed to a radiology department. To do so, the stored instructions for the request resolution method 100 are executable by the at least one electronic processor 20 to implement, on one or more radiology workstations 18, the radiology reading environment 30 via which radiology images are displayed on the radiology workstation and via which a radiology report is received via the radiology workstation.

At 104, the interpreter module 34 is configured to classify the communication requests 31. In some embodiments, the classification of the communication requests 31 includes classifying communication requests on the basis of intended recipient (i.e., radiologist).

At 106, the scheduler module 36 is configured to assign the classified communication requests 31 to agent queues of a plurality of agent queues 40 based on at least the classifications of the communication requests. In some examples, the plurality of agent queues 40 include at least one radiology workstation call queue (e.g., the user interface 42) whose corresponding request resolution agent comprises a user interface (e.g., the telephonic interface 44′ or the text messaging user interface 44″) to a radiology workstation 18 of the one or more radiology workstations. In other examples, the plurality of agent queues 40 further includes at least one automated agent queue whose corresponding request resolution agent comprises the automated request resolution agent (e.g., the automated query queue 46′ or the AI-enabled dialogue window queue 46″).

In some embodiments, when the at least one radiology workstation call queue 42 (e.g., the telephonic interface 44′, the text messaging user interface 44″, the automated query queue 46′ or the AI-enabled dialogue window queue 46″) includes a plurality of radiology workstation call queues (i.e., more than one of the queue options) for different intended recipients. In this embodiment, the radiology workstation call queues 42 are dynamically configured on the basis of logins to the one or more radiology workstations 18. For example, if a radiologist is logged into a first workstation 18 (i.e., workstation A), then the radiology workstation call queue 42 is configured to deliver communication requests 31 to that particular radiologist. If the radiologist logs out of workstation A, and a second, different radiologist logs in to workstation A, then the radiology work station all queue 42 for workstation A is then reconfigured to deliver the communication requests 31 to the second radiologist. This approach can also apply for different query “pools” (e.g., an MM query pool, a CT query pool, and so forth).

At 108, the dispatcher module 38 is configured to route the communication requests 31 assigned to each agent queue 40 to a request resolution agent 50 corresponding to the agent queue. For example, the communication requests 31 are dispatched by the dispatcher module 38 to the simple knowledge base query 52, the AI-enabled dialog expert system 56, or the human radiologist 58.

At 110, the AI optimization module 54 is configured to update the schedule of the communication requests 31 based on an availability of a radiologist, preferences of a radiologist, or if the at least one communication request specifies a specific radiologist. The optimization module 54 is configured to monitor processing of the communication requests 31 by the request resolution agents; and (ii) update the forecasting data of the statistical forecasting knowledge database 52 on the basis of the monitored processing of the communication requests by the request resolution agents 50.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A radiology request monitoring system, comprising:
at least one electronic processor;
a non-transitory computer readable medium storing instructions executable by the at least one electronic processor, the instructions including:
instructions implementing a radiology reading environment via which radiology images are displayed and via which a radiology report is received;
instructions implementing a communication requests interface configured to intercept communication requests directed to a user of the radiology reading environment;
instructions implementing an interpreter module configured to classify the intercepted communication requests;
instructions implementing a scheduler module configured to assign the communication requests to agent queues of a plurality of agent queues based on at least the classifications of the communication requests; and
instructions implementing a dispatcher module configured to route the communication requests assigned to each agent queue to a request resolution agent corresponding to the agent queue.

2. The radiology request monitoring system of claim 1, wherein the scheduler module is configured to combine the classifications of the communication requests with forecasting data from a knowledge database to schedule the routing of the communication requests by the dispatcher module;
wherein the knowledge database is configured to store the forecasting data including one or more of: current queue lengths for the queues of the plurality of queues, expected wait times for the queues of the plurality of queues, and historical wait times for the queues of the plurality of queues.

3. The radiology request monitoring system of claim 2, wherein:
the scheduler module is configured to retrieve information from the communication requests and information from the database related to the communication requests; and to present the retrieved information and the communication requests on a display device.

4. The radiology request monitoring system of claim 2 wherein the instructions further include:
instructions implementing an artificial intelligence (AI) optimization module configured to monitor processing of the communication requests by the request resolution agents and to update the forecasting data of the knowledge database on the basis of the monitored processing of the communication requests by the request resolution agents.

5. The radiology request monitoring system of claim 1, wherein the interpreter module is configured to assign the classifications to the communication requests at least based on natural language processing of natural language content of the communication requests.

6. The radiology request monitoring system of claim 1, wherein the interpreter module is configured to assign the classifications as values for attributes of an attribute vector; wherein attributes of the attribute vector include one or more of:
one or more caller attributes describing the caller;
one or more request type attributes describing the type of request including a request attribute pertaining to a current examination, and a previously prepared radiology report;
a timestamp attribute storing a time when the communication request was intercepted;
a patient attribute identifying a patient to which the communication request pertains;
at least one request content attribute indicating one or more of anatomy, imaging modality, or reason for exam content of the communication request;
an interruption complexity attribute whose value is determined based at least on a number of questions being asked in the communication request.

7. The radiology request monitoring system of claim 1, wherein the plurality of agent queues includes at least one user interface queue whose corresponding request resolution agent comprises a user interface with the user of the radiology reading environment, the at least one user interface queue including one or more of:
a telephonic user interface with the user of the radiology reading environment; and
a text messaging user interface with the user of the radiology reading environment.

8. The radiology request monitoring system of claim 1, wherein the plurality of agent queues further includes at least one automated agent queue whose corresponding request resolution agent comprises an automated request resolution agent the at least one automated agent queue including at least one of:
an automated query queue whose corresponding request resolution agent comprises an expert system retrieving responses from a technical knowledge database; and
an AI-enabled dialogue window queue whose corresponding request resolution agent comprises an AI-enabled dialog expert system interfacing with the technical knowledge database.

9. The radiology workstation of claim 4, wherein the radiology reading environment provides a communication configuration user interface via which the user of the radiology reading environment configures communication preferences relating to a maximum number of communication requests allowed per unit time, and prioritization of communication requests on the basis of request content including one or more of type of modality and type of anatomy.

10. The radiology request monitoring system of claim 9, wherein the AI optimization module is further configured to:
determine a number of available radiologists based on an availability of a radiologist, preferences of a radiologist, or if the at least one communication request specifies a specific radiologist;
determine a maximum time period that a radiologist may be unavailable based on availability of other radiologists and a number of communication requests.

11. The radiology request monitoring system of claim 1, wherein the scheduler module is further configured to:
cluster trends in data contained in the communication requests; and
upload the clustered trends to the dispatcher module to route the communication requests to a specific request resolution agent.

12. A non-transitory computer readable medium storing instructions executable by at least one electronic processor to perform a request resolution method, the method including:
intercepting communication requests directed to a radiology department;
classifying the communication requests;
assigning the communication requests to agent queues of a plurality of agent queues based on at least the classifications of the communication requests; and
routing the communication requests assigned to each agent queue to a request resolution agent corresponding to the agent queue.

13. The non-transitory computer readable medium of claim 12, wherein:
the stored instructions are further executable by the at least one electronic processor to implement, on one or more radiology workstations, a radiology reading environment via which radiology images are displayed on the radiology workstation and via which a radiology report is received via the radiology workstation; and
the plurality of agent queues include at least one radiology workstation call queue whose corresponding request resolution agent comprises a user interface to a radiology workstation of the one or more radiology workstations; wherein the user interface to the radiology workstation comprises at least one of a telephonic interface and a text messaging interface.

14. The non-transitory computer readable medium of claim 12, wherein the plurality of agent queues further includes:
at least one automated agent queue whose corresponding request resolution agent comprises an automated request resolution agent.

15. The non-transitory computer readable medium of claim 12, wherein:
the classification of the communication requests includes classifying communication requests on the basis of intended recipient; and
the at least one radiology workstation call queue includes a plurality of radiology workstation call queues for different intended recipients;
wherein the radiology workstation call queues are dynamically configured on the basis of logins to the one or more radiology workstations.

16. The non-transitory storage medium of claim 12, wherein the stored instructions further include an AI optimization module configured to update a schedule of requests based on an availability of a radiologist, preferences of a radiologist, or if the at least one communication request specifies a specific radiologist.

17. The non-transitory storage medium of claim 16, wherein the AI optimization module is further configured to:
determine a number of available radiologists based on an availability of a radiologist, preferences of a radiologist, or if the at least one communication request specifies a specific radiologist;
determine a maximum time period that a radiologist may be unavailable based on availability of other radiologists and a number of communication requests.

18. The non-transitory storage medium of claim 12, wherein the method further includes:
clustering trends in data contained in the communication requests; and
route the clustered trends to a specific request resolution agent.

19. The non-transitory storage medium of claim 12, wherein the method further includes:
retrieving information from the communication requests and information from a database related to the communication requests; and
presenting the retrieved information and the communication requests on a display device

20. A request resolution method, comprising:
implementing a radiology reading environment via which radiology images are displayed on a display device of a workstation and via which a radiology report is received via one or more user input devices of the workstation;
intercepting, with a communication requests interface, communication requests directed to a user of the radiology reading environment;
classifying, with an interpreter module, the intercepted communication requests;
assigning, with a scheduler module, the communication requests to agent queues of a plurality of agent queues based on at least the classifications of the communication requests, routing, with a dispatcher module, the communication requests assigned to each agent queue to an AI-enabled dialog expert system corresponding to the agent queue; and
monitoring, with an AI optimization module, processing of the communication requests by the AI-enabled dialog expert system.
